(19) United States
(12) Patent Application Publication
(10) Pub. No.: US 2017/0150942 A1
(43) Pub. Date: Jun. 1, 2017

(54) ULTRASONIC DIAGNOSTIC APPARATUS

(71) Applicant: Toshiba Medical Systems Corporation, Otawara-shi (JP)

(72) Inventors: Fuminori FUJITA, Nasushiobara (JP); Kentaro TSUZUKI, Nasushiobara (JP)

(73) Assignee: Toshiba Medical Systems Corporation, Otawara-shi (JP)

(21) Appl. No.: 15/355,282

(22) Filed: Nov. 18, 2016

(30) Foreign Application Priority Data
Dec. 1, 2015 (JP) 2015-234622

(51) Int. Cl. A61B 8/08; A61B 90/00; A61B 8/00

(52) U.S. Cl. CPC A61B 8/0841; A61B 8/4444; A61B 8/488; A61B 8/5207; A61B 8/461; A61B 90/39; A61B 2090/3925

(57) ABSTRACT

An ultrasonic diagnostic apparatus according to a present embodiment includes: an ultrasonic probe attached to a puncture adaptor including puncture holes; and a processing circuitry configured to (A) set a target site of puncturing within an object, the target site of puncturing being specified in an ultrasonic image based on data detected by the ultrasonic probe, (B) identify a puncture hole out of the puncture holes, the puncture hole being at a position corresponding to a position of the target site of puncturing, (C) generate projection information regarding the identified puncture hole, and (D) control a projection apparatus that optically projects information to control the projection information to be projected onto the puncture adaptor.

### CROSS-REFERENCE TO RELATED APPLICATION

This application is based upon and claims the benefit of priority from Japanese Patent Application No. 2015-234622, filed on Dec. 1, 2015, the entire contents of which are incorporated herein by reference.

### FIELD

An embodiment as an aspect of the present invention relates to an ultrasonic diagnostic apparatus.

### BACKGROUND

Recently, for the treatment of prostate cancer, brachytherapy has been practiced in which treatment of a tumor site is performed by radiating a radioactive ray to a tumor site of prostate cancer from a radioactive ray source placed in a prostate gland. In brachytherapy, an ultrasonic probe is inserted into a rectum to acquire an ultrasonic image of a prostate which is adjacent to the rectum. An operator refers to the ultrasonic image to decide at which position in the prostate the radioactive ray source is to be placed. Then, the operator performs puncturing to place the radioactive ray source at a desired position in the prostate while the ultrasonic image is being displayed. When performing puncturing, the operator is enabled to stably perform puncture operation by performing puncture via a puncture adaptor which is fixed to an ultrasonic probe.

The puncture adaptor includes puncture holes and plays a role of guiding a puncture needle by fixing an insertion angle of the puncture needle at each puncture hole.

Typically, position information based on symbols such as figures and alphabets for identifying the position information of each puncture hole is printed on the puncture adaptor, and identification of a puncture hole for guiding to a desired puncture position is performed by an assistant other than the operator notifying of the position information of the puncture hole to be punctured the operator.

However, since multiple puncture holes are disposed on the puncture adaptor, there is a risk that the operator mistakes a puncture position. Moreover, there is also possibility that the operator mishears position information of the puncture hole notified by an assistant.

Since a puncture adaptor is used during surgery, it must always be kept hygienic. Although a conventional art discloses a technique to notify of a puncture hole to be punctured the operator by lighting a light source disposed on a puncture adaptor, a problem exists in that disinfection and sterilization of the puncture adaptor is time consuming. Moreover, it is desirable that the puncture adaptor is disposal.

An objective to be achieved by the present invention is to provide an ultrasonic diagnostic apparatus which is capable of projecting and displaying the position of a desired puncture hole at the time of puncturing onto a puncture adaptor.

### BRIEF DESCRIPTION OF THE DRAWINGS

In accompanying drawings,

FIG. 1 is a block diagram showing an outline configuration of an ultrasonic diagnostic apparatus according to a first embodiment;

FIG. 2 is an external view showing an example of a puncture adaptor according to the first embodiment;

FIG. 3 is an external view showing an example of projection display onto the puncture adaptor by the ultrasonic diagnostic apparatus according to the first embodiment;

FIG. 4 is an external view showing an outline of puncture treatment by the ultrasonic diagnostic apparatus according to the first embodiment;

FIG. 5 is a flowchart showing an example of puncture treatment according to the first embodiment;

FIG. 6 is a flowchart showing an example of puncture treatment by an ultrasonic diagnostic apparatus according to a second embodiment;

FIG. 7 is an external view showing an example of projection display onto a puncture adaptor by the ultrasonic diagnostic apparatus according to the second embodiment;

FIG. 8 is a flowchart showing an example of puncture treatment by an ultrasonic diagnostic apparatus according to a third embodiment;

FIG. 9 is an external view showing an example of projection display onto the puncture adaptor by the ultrasonic diagnostic apparatus according to the third embodiment;

FIG. 10 is a block diagram showing an outline configuration of an ultrasonic diagnostic apparatus according to a fourth embodiment;

FIG. 11 is a flowchart showing an example of warning notification by the ultrasonic diagnostic apparatus according to the fourth embodiment; and

FIG. 12 is a diagram showing an example of a puncture adaptor and an ultrasonic probe included in an ultrasonic diagnostic apparatus according to a fifth embodiment.

### DETAILED DESCRIPTION

Hereinafter, embodiments will be described with reference to the drawings.

The ultrasonic diagnostic apparatus according to the present embodiment includes: an ultrasonic probe attached to a puncture adaptor including puncture holes; and a processing circuitry configured to (A) set a target site of puncturing within an object, the target site of puncturing being specified in an ultrasonic image based on data detected by the ultrasonic probe, (B) identify a puncture hole out of the puncture holes, the puncture hole being at a position corresponding to a position of the target site of puncturing, (C) generate projection information regarding the identified puncture hole, and (D) control a projection apparatus that optically projects information to control the projection information to be projected onto the puncture adaptor.

### 1. First Embodiment

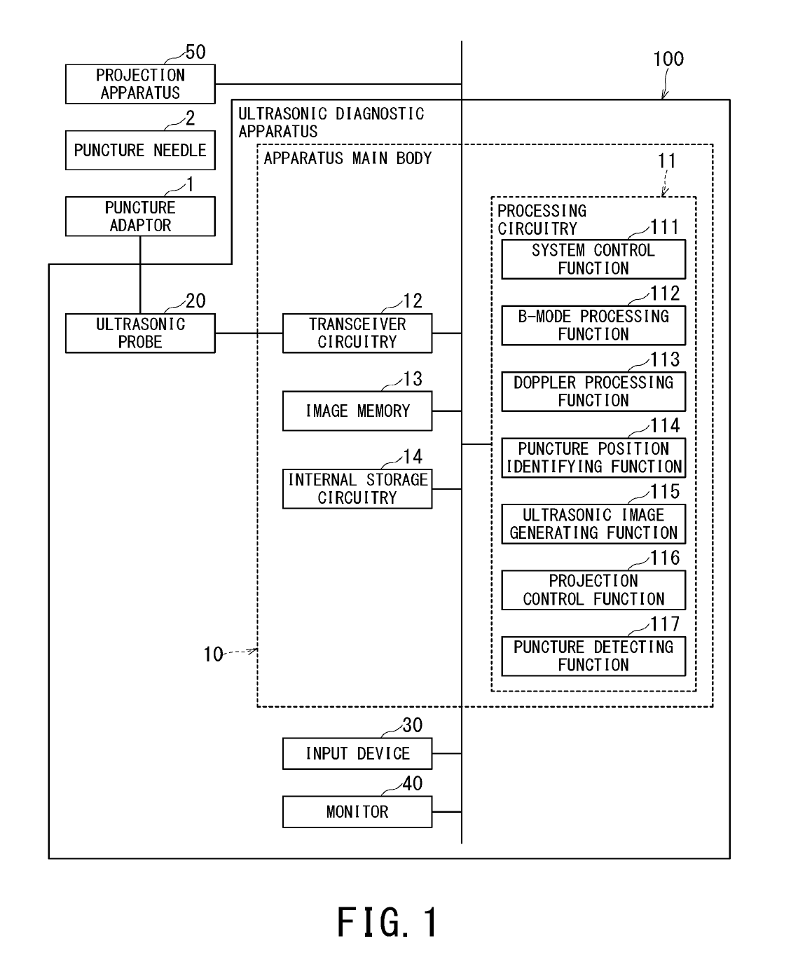

A configuration of an ultrasonic diagnostic apparatus 100 according to a first embodiment will be described with reference to a block diagram of FIG. 1.

FIG. 1 is a block diagram showing an outline configuration of the ultrasonic diagnostic apparatus 100 according to the first embodiment.

The ultrasonic diagnostic apparatus 100 includes an apparatus main body 10, an ultrasonic probe 20, an input device 30, and a monitor 40.

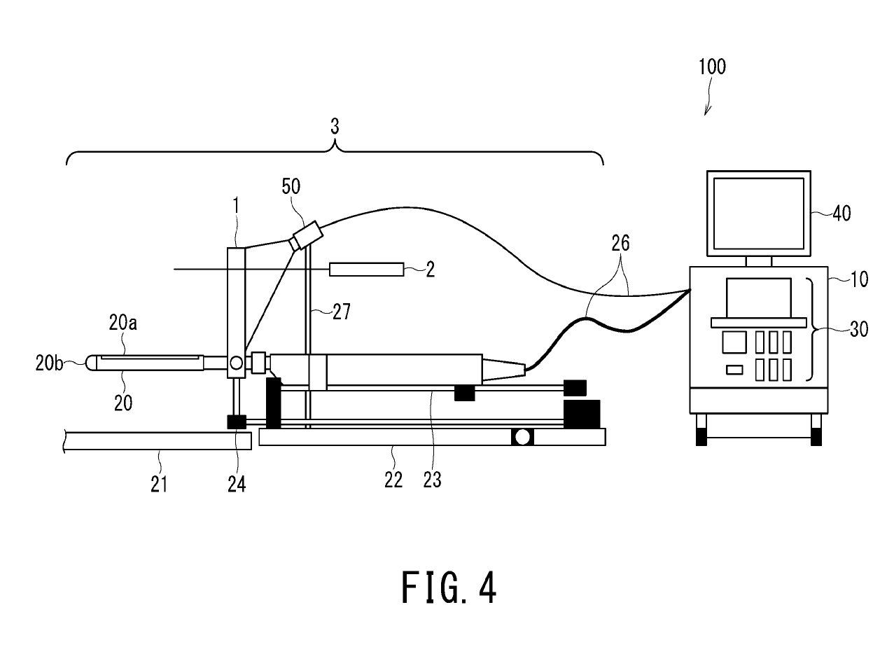

The ultrasonic probe 20 is detachably connected to the apparatus main body 10 which is to be described later. The ultrasonic probe 20 receives a drive signal supplied from transceiver circuitry 12 included in the apparatus main body 10 and generates an ultrasonic wave to transmit it to an object. Moreover, the ultrasonic probe 20 receives a reflected wave from the object and converts it into an electric signal to output it to the transceiver circuitry 12. Upon an ultrasonic wave been transmitted to an object from the ultrasonic probe 20, the transmitted ultrasonic wave is reflected at a discontinuity plane of acoustic impedance in body tissue of the object, and the reflected wave is received at the ultrasonic probe 20 as a reception signal. The amplitude of a reflected wave to be received depends on the difference in acoustic impedance at a discontinuity plane at which the ultrasonic wave is reflected. The structure of the ultrasonic probe 20 will be described in detail in the description of FIG. 4.

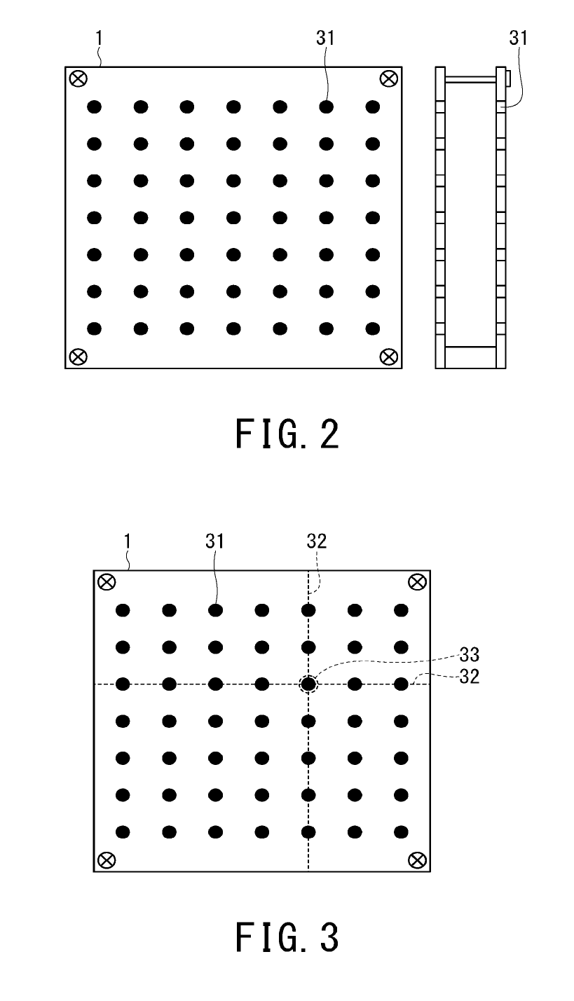

A puncture adaptor 1 will be described by using FIG. 2. FIG. 2 is an external view showing an example of the puncture adaptor 1 according to the first embodiment. To be specific, FIG. 2 illustrates a front view and a side view of the configuration of the puncture adaptor 1.

The puncture adaptor 1 is a puncture guide to be used as a guide by an operator when performing puncturing. The puncture adaptor 1 includes puncture holes 31 arranged in a grid pattern for piercing a puncture needle 2 into a target site of puncturing in the object. The “target site of puncturing” used in the present embodiment is defined as a site within the object, the site being specified in advance by an operator, for example, by referring to an ultrasonic image. For example, target sites of puncturing are set in the surrounding of the prostate gland since when performing brachytherapy of prostate cancer, radiation sources are disposed so as to surround the prostate gland or a tumor site of prostate cancer.

As shown in FIG. 2, puncture holes 31 are disposed in the puncture adaptor 1, and the operator controls the puncture needle 2 so as to insert the puncture needle 2 into a desired puncture hole on the puncture adaptor 1. The puncture adaptor 1 has for example a structure in which two plates each of which has puncture holes 31 are attached to each other so as to be spaced apart as shown in FIG. 2. As a result of passing a puncture hole 31 which is common to the two plates, the angle upon insertion of the puncture needle 2 is fixed, allowing the puncture needle 2 to be pierced along the body axis direction of the object. Moreover, the puncture adaptor 1 is desirably made of a biocompatible material assuming that the puncture adaptor 1 comes into contact with the object.

Note that the shape of the puncture adaptor 1, the number of the puncture holes 31, the pitch of the puncture holes 31, and the positional relationship of each puncture hole 31 with respect to an imaging position of the ultrasonic probe 20 differ depending on the type of the puncture adaptor 1. These information, that is, the information regarding the shape of the puncture adaptor 1, the number of the puncture holes 31, the pitch of the puncture holes 31, and the like is associated with information to indicate the type of the puncture adaptor 1 and a model number of the puncture adaptor 1, and stored in internal storage circuitry 14 in the ultrasonic diagnostic apparatus 100.

A projection apparatus 50 of FIG. 1, which is connected with the ultrasonic diagnostic apparatus 100, includes a projector, a laser pointer which is capable of pointing at a puncture hole 31, or the like. The projection apparatus 50 may have a configuration which allows projection information to be projected onto the puncture adaptor 1. The projection information is information regarding a specific puncture hole 31 in the puncture adaptor 1. In addition to information regarding the puncture position, number of times of puncturing, and a puncture hole 31 in which puncturing is finished may be projected.

The input device 30 (input section), which includes a mouse, a key board, a button, a panel switch, a touch command screen, a foot switch, a track ball, and the like, accepts various setting requests from the operator, and transfers the accepted various setting requests to the apparatus main body 10. For example, setting of the target site of puncturing and the type of the puncture adaptor 1 is performed by an input from the operator.

The monitor 40 is a display device for displaying a GUI (Graphical User Interface) which is used by the operator for inputting various setting requests by using the input device 30, an ultrasonic image generated at the apparatus main body 10, and the like, and is made up of, for example, a liquid crystal display.

The apparatus main body 10 is an apparatus that reads reflected waves received by the ultrasonic probe 20 to generate an ultrasonic image, and includes processing circuitry 11, transceiver circuitry 12, an image memory 13, and internal storage circuitry 14.

The transceiver circuitry 12 (transmission/reception section) includes in combination a pulser circuit, a delay circuit, a trigger generation circuit, and the like, and supplies a driving signal to the ultrasonic probe 20. The pulser circuit repeatingly generates a rate pulse for forming a transmission ultrasonic wave at a predetermined rate frequency. Moreover, the delay circuit gives a delay time for each piezoelectric transducer, which is needed for focusing ultrasound generated from the ultrasonic probe 20 into a beam shape and determining transmission directivity, to each rate pulse generated by the pulser circuit. Moreover, the trigger generation circuit applies a driving signal (driving pulse) to the ultrasonic probe 20 at a timing corresponding to the rate pulse. That is, the delay circuit arbitrary adjusts the transmission direction of ultrasound from the piezoelectric transducer plane by varying delay time to be given to the each rate pulse.

Note that the transceiver circuitry 12 has a function to read an instruction from a system control function 111 of the processing circuitry 11 to be described later, and is instantly able to change the transmission frequency, the transmission drive voltage, and the like for performing a predetermined scan sequence. Especially, changing of the transmission drive voltage is realized by a linear amplifier type sending circuit which is instantly able to switch its value, or a mechanism for electrically switching power supply units.

The transceiver circuitry 12 further includes an amplifier circuit, an A/D converter, an adder, and the like, and performs various processing on reflected wave signals received by the ultrasonic probe 20 to generate reflected wave data. The amplifier circuit amplifies the reflected wave signal for each channel to perform gain correction processing. The A/D converter performs A/D conversion of the reflected wave signal which has been gain corrected, and gives it a delay time necessary for determining reception directivity. The adder performs addition processing of the reflected wave signal corresponding to the given delay time to generate reflected wave data. By the addition processing of the adder, the reflection component from the direction according to the reception directivity of the reflected wave signal is emphasized.

In this way, the transceiver circuitry 12 controls the transmission directivity and the reception directivity in the transmission/reception of ultrasound. Note that the transceiver circuitry 12 has a function of instantly changing delay information, transmission frequency, transmission driving voltage, number of opening elements, and the like by the control from the system control function 111.

The image memory 13 is a memory that stores ultrasonic images generated by the ultrasonic image generating function 115 of the processing circuitry 11 to be described later, and projection information generated by the projection control function 116 of the processing circuitry 11 to be described below.

The internal storage circuitry 14 (internal storage section) stores various data such as control programs for performing ultrasound transmission/reception, image processing and display processing, and information regarding diagnostic information (for example, patient ID, opinions of doctor), diagnostic protocols, various body marks, and the puncture adaptor 1.

The processing circuitry 11 (processing section) performs various processing relating to the ultrasonic diagnostic apparatus 100. The processing circuitry 11 includes a system control function 111, a B-mode processing function 112, a Doppler processing function 113, a puncture position identifying function 114, an ultrasonic image generating function 115, a projection control function 116, and a puncture detecting function 117.

The system control function 111 (system control section) controls the entire processing in the ultrasonic diagnostic apparatus 100. To be specific, the system control function 111 controls the transceiver circuitry 12, and the B-mode processing function 112, the Doppler processing function 113, the ultrasonic image generating function 115, and the projection control function 116, which are to be described later, by using various setting requests inputted from the operator via the input device 30, and various control programs and various data read from the internal storage circuitry 14.

The B-mode processing function 112 (B-mode processing section) receives reflected wave data from the transceiver circuitry 12, and performs logarithmic amplification, envelop detection processing, and the like on the received reflected wave data to generate data (B-mode data) in which signal strength is represented by brightness of luminance.

The Doppler processing function 113 (Doppler processing section) receives reflected wave data from the transceiver circuitry 12, performs frequency analysis of velocity information from the received reflected wave data, and extracts echo components of blood flow, tissue, contrast agent, due to Doppler effect to generate data (Doppler data) which are obtained by extracting moving body information such as average velocity, variance, and power extracted at multiple points.

The puncture position identifying function 114 (puncture position identification section) identifies the puncture hole 31 on the puncture adaptor 1 corresponding to the target site of puncturing from the ultrasonic image generated by the ultrasonic image generating function 115.

First, preprocessing for identifying the puncture hole 31 will be described.

The projection control function 116 to be described later reads an ultrasonic image generated by the ultrasonic image generating function 115 from the image memory 13 to cause it to be displayed on the monitor 40. At this moment, first, a linear image including a prostate site will be displayed on the monitor 40. Next, an acquisition position of a convex image for performing the setting of the target site of puncturing on the linear image is specified by the operator.

When performing brachytherapy of prostate cancer, it is necessary to dispose radiation sources so as to surround the prostate gland of the object. For that reason, it is necessary to acquire convex images of cross sections corresponding to the size of prostate gland and the size of tumor site with respect to the depth direction. When acquisition point of each convex image is specified in a linear image, the ultrasonic probe 20 moves and acquires a convex image at the specified position. In this situation, the ultrasonic probe 20 may be moved to the predetermined position by the operator, or may be moved to the predetermined position by motor driving, or the like. Each convex image is successively displayed on the monitor 40, and the target site of puncturing is specified in the convex image by the operator. The information regarding the position coordinates of the specified target site of puncturing on the convex image, and the acquisition position of the convex image on the linear image is stored on the internal storage circuitry 14.

Next, the puncture position identifying function 114 identifies the puncture hole 31 on the puncture adaptor 1 corresponding to the target site of puncturing specified by the operator. To be more specific, the puncture position identifying function 114 reads the position information of the target site of puncturing on the convex image set by the operator. Next, the puncture position identifying function 114 converts the position information on the convex image into mechanical coordinates which are represented by horizontal and vertical distances from the ultrasonic probe. The puncture position identifying function 114 reads a table which shows correspondence between the mechanical coordinates stored in the internal storage circuitry 14 and each puncture hole 31 on the puncture adaptor 1, and determines the puncture hole 31 corresponding to the set target site of puncturing.

Note that the position of a target site of puncturing set by the operator for an ultrasonic image not necessarily completely coincides with the position of a puncture hole 31 on the puncture adaptor 1. In such a case, it is also possible to automatically select a puncture hole 31, whose distance from the target site of puncturing set on an ultrasonic image is shortest. Alternatively, it may be arranged such that with markers corresponding to the positions of puncture holes 31 being displayed on an ultrasonic image displayed on the monitor 40, the position of the target site of puncturing specified by the operator is made to coincide with the position of a puncture hole 31 by the operator selecting the target site of puncturing among the markers.

The puncture position identifying function 114 becomes able to dispose radiation sources so as to surround a prostate gland by performing a series of processing described so far on all the convex images in each slice plane.

The above described puncture position identifying function 114 is an example of the setting section for setting the target site of puncturing. Although, in the present embodiment, description has been made assuming that first a linear image of a site including the prostate gland of the object is acquired, and the target site of puncturing is set from each convex image of a position specified in the linear image, the setting method of the target site of puncturing will not be limited to the above described mode. For example, when the target site of puncturing is set, a linear image not necessarily needs to be acquired, and it may be arranged to specify the target site of puncturing with reference to the convex image alone, and change the acquisition position of the convex image by moving the ultrasonic probe 20.

The ultrasonic image generating function 115 (ultrasonic image generation section) generates an ultrasonic image from the data generated by the B-mode processing function 112 and the Doppler processing function 113. That is, the ultrasonic image generating function 115 generates a B-mode image, in which the intensity of reflected wave is represented by luminance, from the B-mode data generated by the B-mode processing function 112. Moreover, the ultrasonic image generating function 115 generates an average velocity image, a variance image, or a power image, each of which represents moving body information, or a color Doppler image which is a combination image thereof, from the Doppler data generated by the Doppler processing function 113.

Further, the ultrasonic image generating function 115 is also able to generate a synthetic image in which character information of various parameters, scale marks, body marks, or the like is synthesized with an ultrasonic image. Moreover, the ultrasonic image generating function 115 may have a function of causing information regarding various parameters etc. relating to puncturing to be superimposed and displayed on the ultrasonic image, and thus to be displayed on the monitor 40. For example, the ultrasonic image generating function 115 may be configured such that the target site of puncturing set by the operator is displayed on an ultrasonic image, or the number of radiation sources to be placed within the object by puncturing using the same puncture hole 31 is superimposed and displayed.

The projection control function 116 (projection control section) displays the position of the puncture hole 31 corresponding to the target site of puncturing on the ultrasonic image generated by the ultrasonic image generating function 115, and generates projection information for supporting puncturing. The projection information is information regarding a specific puncture hole 31 in the puncture adaptor 1 as the puncture position. In addition to the information regarding puncture position, number of times of puncturing, and the puncture hole 31 for which puncturing is finished may be projected. The projection information may display all the puncture holes 31 corresponding to target sites of puncturing, or projection information regarding the puncture hole corresponding to the target site of puncturing may be projected successively for each puncture hole 31.

The image data of projection information generated by the projection control function 116 is transmitted to the projection apparatus 50, and the projection information is displayed on the puncture adaptor 1 via the projection apparatus 50. Moreover, the projection control function 116 performs control to finish projection. The projection control function 116 has a function of performing switching of the projection information to be projected onto the puncture adaptor 1 upon detection that puncturing is performed by the puncture detecting function 117 to be described later. For example, projection regarding the puncture hole 31 for which puncturing is finished is finished, and projection information regarding another puncture hole is projected. The projection control function 116 is one example of the projection information generation section which generates projection information regarding puncture position. Moreover, the projection control function 116 may have a display control function of not only controlling the projection information onto the puncture adaptor 1, but also controlling the information to be displayed on the monitor 40 at the same time.

FIG. 3 is an external view showing an example of projection display onto the puncture adaptor 1 by the ultrasonic diagnostic apparatus 100 according to the first embodiment.

For example, two or more line segments 32 which intersect at a puncture hole 31 are displayed together with the puncture holes 31 corresponding to the target sites of puncturing on the puncture adaptor 1, and the intersection point of the two or more line segments 32 is displayed as a puncture hole 33 which is going to be the puncture target. Moreover, grids are displayed on the puncture adaptor 1, and only the line segment 32 which includes the puncture hole 33 which is going to be the puncture target may be displayed with emphasis. Further, only the puncture hole 33 of puncture target may be displayed with emphasis as one point.

The puncture detecting function 117 (puncture detection section) detects whether or not puncturing is performed through a puncture hole 31 corresponding to the target site of puncturing within the object. To be more specific, the puncture detecting function 117 reads an acquisition position of each convex image and position coordinates of each target site of puncturing on a linear image from the internal storage circuitry 14. Upon detecting that after the operator pierced the puncture needle 2 into the object, a radiation source or the puncture needle 2 has been imaged at the position of each target site of puncturing on the convex image, the puncture detecting function 117 recognizes that the radiation source is placed at the target site of puncturing, and detects that the puncturing has been performed.

As a method for detecting a radiation source or a puncture needle 2 on an ultrasonic image, puncture detecting function 117 detects changes in luminance value of image, or the like. Moreover, the puncture detecting function 117 detects whether or not the radiation source is placed at the target site of puncturing on each convex image at a different slice position. In this situation, for example, upon confirming that the radiation source has been placed on a convex image at a specific slice position by the puncture detecting function 117, the operator changes acquisition position of convex image by the ultrasonic probe 20, and causes the convex image at a next slice position to be displayed on the monitor 40 via the input device 30, thereafter repeating the above described processing on each puncture hole 31.

Each processing function performed in each component of the processing circuitry 11, which includes the system control function 111, the B-mode processing function 112, the Doppler processing function 113, the puncture position identifying function 114, the ultrasonic image generating function 115, the projection control function 116, and the puncture detecting function 117, is recorded in the internal storage circuitry 14 in a form of a computer executable program. The processing circuitry 11 is a processor that reads programs from the storage circuit and executes them to realize a function corresponding to each program. In other words, the processing circuitry 11 in a state of having read out each program, will have each function shown in the processing circuitry 11 of FIG. 1. Note that although it has been described that the processing functions performed in the system control function 111, the B-mode processing function 112, the Doppler processing function 113, the puncture position identifying function 114, the ultrasonic image generating function 115, the projection control function 116, and the puncture detecting function 117 may be realized in the processing circuitry 11 comprising a single circuit, the single circuit executing a program. Alternatively, the functions 111-117 may be realized in the processing circuitry 11 comprising multiple independent circuits, the independent circuits executing parts of the program, respectively.

FIG. 4 is an external view showing an outline of puncture treatment by the ultrasonic diagnostic apparatus 100 according to the first embodiment.

The puncture apparatus 3 shown in FIG. 4 has a configuration which is separated from the apparatus main body 10, and is connected with the apparatus main body 10 via a cable 26. The puncture apparatus 3 includes a puncture adaptor 1, a puncture needle 2, an ultrasonic probe 20, a bed 21, a puncture apparatus fixing base 22, an ultrasonic probe supporting mechanism 23, a puncture adaptor supporting mechanism 24, and a projection apparatus 50.

The puncture adaptor 1 is fixed to the puncture apparatus fixing base 22 via the puncture adaptor supporting mechanism 24. The operator is able to select a desired puncture hole 31 out of the puncture holes 31 of the puncture adaptor 1, and pierce the puncture needle 2 into the target site of puncturing. Moreover, radioactive substance is attached to a distal end of the puncture needle 2, and the operator places the radioactive substance at the distal end at a predetermined position of the object at the time of puncturing.

The ultrasonic probe 20 includes multiple piezoelectric transducers, and ultrasound is generated by the vibration of these piezoelectric transducers. In the present embodiment, the ultrasonic probe 20 includes a first piezoelectric transducer 20a which acquires a linear image which is an ultrasonic image of a sagittal plane of the object, and a second piezoelectric transducer 20b which acquires a convex image which is an ultrasonic image of an axial plane of the object. The first piezoelectric transducer 20a is attached to a part of a circumferential side face of the ultrasonic probe 20 along a longitudinal direction. The second piezoelectric transducer 20b is attached at a distal end part of the ultrasonic probe 20 along a circumferential direction. The first piezoelectric transducer is able to be used for generating a liner image which shows a sagittal plane of the object, and the second piezoelectric transducer is able to be used for generating a convex image which shows an axial plane of the object.

The bed 21 is made up of a bed on and to which the object is made recumbent and fixed, and the like. The bed 21 has a function of fixing the object such that the positional relationship with the puncture apparatus 3 will not be displaced in a state in which the object is able to be driven in a body axis direction.

The puncture apparatus fixing base 22 is a holder for fixing the ultrasonic probe supporting mechanism 23, the puncture adaptor supporting mechanism 24, and the projection apparatus 50 as shown in FIGS. 2 and 4. The ultrasonic probe supporting mechanism 23 holds the ultrasonic probe 20 such that the ultrasonic probe 20 moves along the body axis direction. The ultrasonic probe supporting mechanism 23, which is connected to the ultrasonic probe 20, is configured to be able to move the ultrasonic probe 20, which is inserted into the object, in the body axis direction and adjusts an imaging position of ultrasonic image. The ultrasonic probe supporting mechanism 23 is made up of a fixing base which is able to move the ultrasonic probe 20 in the body axis direction. The puncture adaptor supporting mechanism 24 holds the puncture adaptor 1. The puncture adaptor supporting mechanism 24 is made up of a fixing base which is able to move the puncture adaptor 1 in the up-and-down direction and the body axis direction of the object. The puncture adaptor supporting mechanism 24 is one example of mounting base for supporting the puncture adaptor 1.

The projection apparatus 50 (projection section) is fixed onto, for example, the puncture apparatus fixing base 22 via a support column 27 in the puncture apparatus 3. Particularly, although the projection apparatus 50 is desirably disposed at a position obliquely upward of the vicinity of the puncture adaptor 1, it may be disposed on a wall surface of the ultrasonic diagnostic apparatus 100 and an inspection room. This makes it possible to prevent projection information to be projected onto the puncture adaptor 1 from being occluded by the operator.

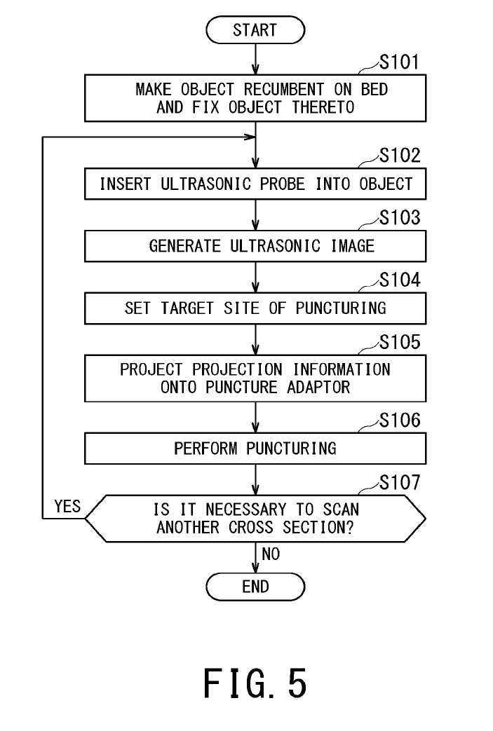

Next, one example of puncture treatment by use of the ultrasonic diagnostic apparatus 100 according to the first embodiment will be described with reference to a flowchart of FIG. 5.

FIG. 5 is a flowchart showing an example of puncture treatment according to the first embodiment.

First, at S101, the object is made recumbent on the bed 21 and is fixed thereto.

Next, at S102, the ultrasonic probe 20 is inserted into, for example, the rectum of the object by the operator, and is adjusted to be a position at which imaging of prostate is possible.

At S103, the ultrasonic probe 20 transmits/receives ultrasound so that the ultrasonic image generating function 115 generates an ultrasonic image. The generated ultrasonic image is stored in the image memory 13, and at the same time, the ultrasonic image acquired by the ultrasonic probe 20 is displayed on the monitor 40.

Next, at S104, the input device 30 accepts input information from the operator, and setting of a target site of puncturing is performed on the ultrasonic image generated at S103. To be more specific, the projection control function 116 reads the ultrasonic image generated at S103 from the image memory 13 to display it on the monitor 40. With reference to the ultrasonic image displayed on the monitor 40, setting of a target site of puncturing is performed via the input device 30 by the operator. The information regarding the set target site of puncturing is transmitted to the puncture position identifying function 114. The puncture position identifying function 114 reads the position information of the target site of puncturing which has been set on an ultrasonic image, and selects a puncture hole 31 on the puncture adaptor 1.

Next, at S105, the projection control function 116 generates projection information to be projected onto the puncture adaptor 1, and the projection apparatus 50 projects the projection information onto the puncture adaptor 1. First, the projection control function 116 generates image data which displays information regarding the puncture holes 31 on the puncture adaptor 1 identified by the puncture position identifying function 114. The generated image data is transmitted to the projection apparatus 50 via the image memory 13. The projection apparatus 50 reads received image data, and projects projection information onto the puncture adaptor 1. In this situation, when the target site of puncturing is set in multiple numbers, the projection information regarding puncture holes 31 is projected at once.

Next, at S106, puncturing is performed by the operator. The operator inserts the puncture needle 2 into desired puncture hole 31 referring to the projection information projected on the puncture adaptor 1. Moreover, the puncture detecting function 117 detects that puncturing is performed at a desired position. Projection of a puncture hole 31 at which performance of puncturing is detected by the puncture detecting function 117 is finished by the projection control function 116. In this situation, when puncturing is successively finished on puncture holes 31 which are projected on the puncture adaptor 1, the projection of the puncture hole 31 is finished. When puncturing is finished on all the puncture holes 31 projected and projection thereof is finished, the process proceeds to step S107.

At step S107, the necessity of scanning of another cross section of the object is determined by the operator. The necessity of scanning of another cross section is determined by the operator. The case in which scanning of another cross section is necessary applies to a case in which the target site of puncturing is included in a cross section different from the cross section which has been scanned in the flow before S106. In this situation, to acquire an ultrasonic image of another cross section, the ultrasonic probe 20 is operated again by the operator to change the scanning position. That is, the flow from S102 is repeated again.

By setting the target site of puncturing in an ultrasonic image by performing a series of processing described above, a desired puncture hole 31 for guiding the puncture needle 2 to the target site of puncturing is indicated, thus allowing to assist the operator.

In the present embodiment, although description has been made on the case in which projection display regarding puncture holes 31 is projected at once, and when puncturing is finished, projection display is successively finished, it may be configured such that the projection information is displayed for each puncture hole 31, and is changed to the projection information regarding next puncture hole 31 every time puncturing is finished.

### 2. Second Embodiment

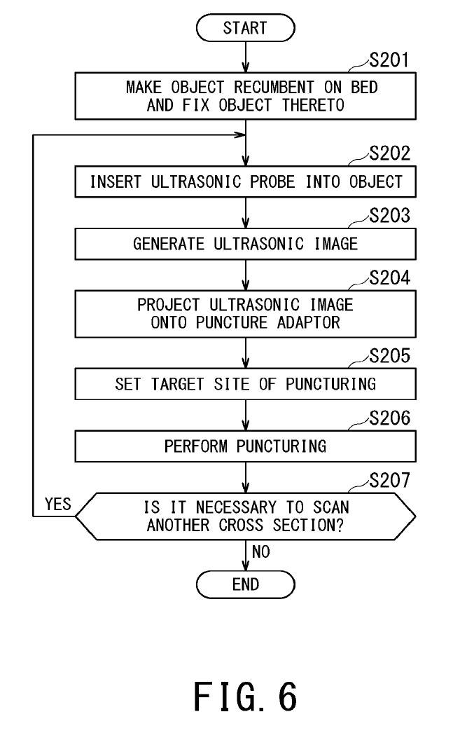

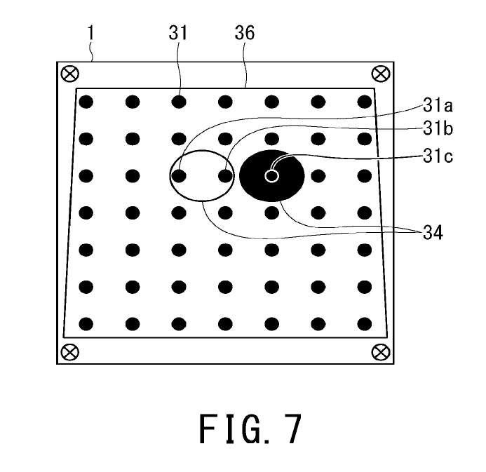

In a second embodiment, in place of the projection information which is projected on the puncture adaptor 1 in the first embodiment, an ultrasonic image including the target site of puncturing which is acquired by the ultrasonic probe 20, for example, a convex image 36 is projected on the puncture adaptor 1 by using the projection apparatus 50 will be described using FIGS. 6 and 7.

FIG. 6 is a flowchart showing an example of puncture treatment by the ultrasonic diagnostic apparatus 100 according to the second embodiment. Since steps from S201 to S203 are configured in the same manner as steps from S101 to S103 of the first embodiment described in FIG. 4, description thereof will be omitted.

At S204, upon acquisition of a convex image 36 at S203, the projection apparatus 50 projects the acquired convex image 36 onto the puncture adaptor 1. To be more specific, the image data of the convex image 36 generated by the ultrasonic image generating function 115 is transmitted to the projection apparatus 50 by the projection control function 116. The projection apparatus 50 reads the received image data and projects the convex image 36 onto the puncture adaptor 1. The convex image 36 to be projected is for example a real time image of a region including the prostate gland of the object.

Next, at S205, confirmation of the target site of puncturing is performed. To be more specific, at S204, the operator refers to the convex image 36 projected onto the puncture adaptor 1, and recognizes a tumor site of the prostate gland. Next, a puncture hole 31 at a position where radiation sources are able to be disposed in such a way to surround the tumor site is recognized by the operator as the puncture hole 31 of puncture target. Moreover, the puncture hole 31 of puncture target may be set to the puncture hole 31 at a position corresponding to the tumor site of the prostate gland.

Since the processing according to from S206 to S207 is of the same configuration as the processing according to from S106 to S107, description thereof will be omitted.

FIG. 7 is an external view showing an example of projection display onto the puncture adaptor by the ultrasonic diagnostic apparatus 100 according to the second embodiment. To be specific, FIG. 7 is a diagram to show the puncture adaptor 1, and one example of display of convex image 36 projected onto the puncture adaptor 1.

The target site of puncturing 34 such as a tumor site and an organ site is displayed on the convex image 36. The operator becomes possible to recognize the puncture hole 31 (in FIG. 7, for example, puncture holes 31a, 31b, 31c) on the puncture adaptor 1 corresponding to the target site of puncturing 34 as the puncture hole 31 to be used at the time of puncturing.

By performing a series of processing described above, it becomes not necessary for the operator to set the target site of puncturing while referring to a monitor, or the like separately, and becomes possible to set the target site of puncturing referring to the convex image 36 projected onto the puncture adaptor 1. In this situation, it may be arranged such that the puncture hole 31 of the puncture adaptor 1, which corresponds to the site which is recognized as the target site of puncturing by the operator, is projected and displayed by the method according to the first embodiment.

Although, in the second embodiment, it has been described that an ultrasonic image is projected onto the puncture adaptor 1, the diagnostic image to be projected will not be limited to an ultrasonic image. For example, it may be arranged to project a diagnostic image acquired by an X-ray image diagnostic apparatus, an X-ray CT (Computed Tomography) apparatus, an MR (Magnetic Resonance) apparatus, or the like, which is another modality. Describing a case in which a diagnostic image acquired by an X-ray CT apparatus is projected, volume data of the object is acquired by the X-ray CT apparatus in advance. Alignment between the acquired volume data of CT and an ultrasonic image is performed to reconstruct an MPR (Multi-Planar Reconstruction) image of a cross section corresponding to the imaging plane of the ultrasonic image from within the volume data of CT. The reconstructed MPR image is projected onto the puncture adaptor 1 by the projection apparatus 50. Moreover, the cross section of the MPR image to be projected and displayed on the puncture adaptor 1 is updated and displayed interlocked with the position of the ultrasonic probe 20.

The ultrasonic image to be projected on the puncture adaptor 1 will not be limited to a stationary image, and the ultrasonic image may be updated in real time. Further, it may be arranged to display, in place of a convex image acquired by the ultrasonic probe 20, a reconstructed image of an arbitrary cross section within the object, which is generated from the acquired volume data by reconstructing the volume data acquired by the ultrasonic probe 20. The arbitrary cross section is preferably a cross section perpendicular to an axial direction of the ultrasonic probe 20, that is, an advancing direction of the puncture needle.

### 3. Third Embodiment

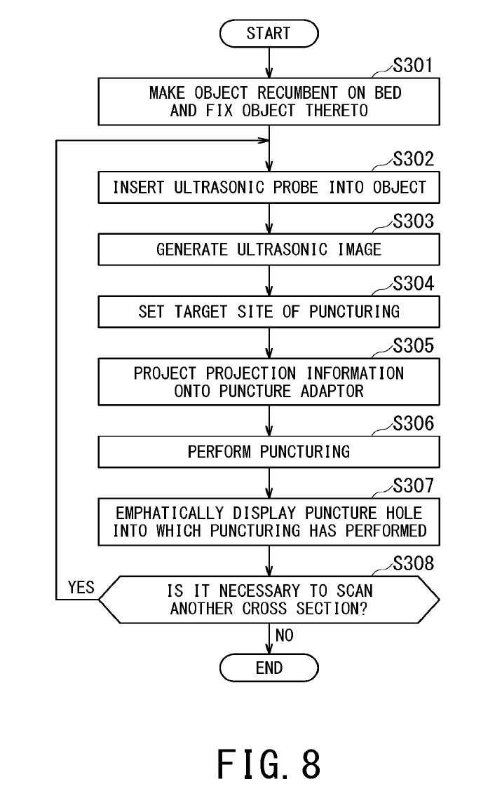

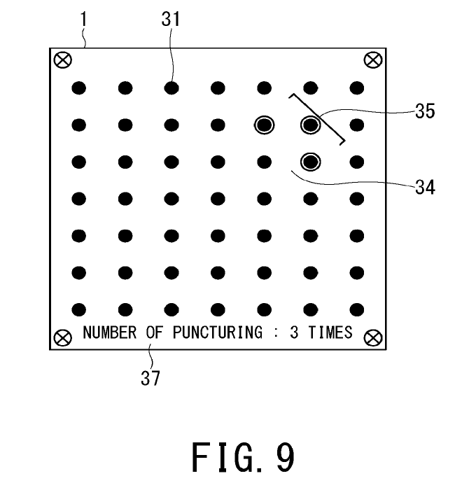

In a third embodiment, description will be made on a case, in which the puncture hole 31 for which puncturing is finished is displayed with emphasis by the projection apparatus 50, by using FIGS. 8 and 9.

FIG. 8 is a flowchart showing an example of puncture treatment by the ultrasonic diagnostic apparatus 100 according to the third embodiment.

In the description regarding the present flowchart, description will be made on a case in which the configuration of the third embodiment is added to the configuration of the first embodiment.

Since the processing according to from S301 to S306 is of the same configuration as the processing according to from S101 to S106 of the first embodiment, description thereof will be omitted.

At S307, when projection of the puncture hole 31 which is projected by the projection apparatus 50 is finished at S306, a puncture hole is displayed with emphasis by projection information different from that of the time of puncturing. To be more specific, when the puncture detecting function 117 determines that puncturing is finished, the puncture detecting function 117 transmits an instruction to finish projection of the projection information of the puncture hole 31 projected and displayed at the time of puncturing to the projection control function 116. Upon determination that projection of projection information is finished, the projection control function 116 switches display so as to emphasize a puncture hole 35 for which puncturing is finished. The processing according to S308 is of the same configuration as the processing according to S107 of the first embodiment, description thereof will be omitted.

FIG. 9 is an external view showing an example of projection display onto the puncture adaptor by the ultrasonic diagnostic apparatus 100 according to the third embodiment. To be specific, FIG. 9 is a diagram to show one example of display when the puncture holes 35 which have been used for puncturing is displayed with emphasis, and projected and displayed on the puncture adaptor 1 by the projection apparatus 50.

The puncture holes 35 which have been used for puncturing are each displayed with emphasis in such a way to surround the circumference thereof, for example. Moreover, as shown in FIG. 9, number of times of puncturing information 37 corresponding to the puncture holes 35 for which puncturings have been finished may be projected onto the puncture adaptor 1. For example, the number of times of puncturing information 37 may display the number of times puncturing has been finished as “number of puncturings: 3 times”, or the like as shown in FIG. 9.

By performing a series of processing described above, it becomes possible that the operator distinguishes and visually recognizes the punctures hole 35 for which puncturings have been finished from the puncture hole 31 for which puncturing is needed on the puncture adaptor 1, thereby reducing a risk regarding the failure of puncturing.

Although the third embodiment has been described taking example of the configuration added to the first embodiment, it may be added to either of the first and second embodiments.

### 4. Fourth Embodiment

In a fourth embodiment, description will be made on a case in which when the operator intends to insert the puncture needle 2 into a puncture hole 31 other than the desired puncture hole 31 on the puncture adaptor 1, the ultrasonic diagnostic apparatus 100 sends a warning. In the fourth embodiment, the ultrasonic diagnostic apparatus 100 includes processing circuitry 11b in place of the processing circuitry 11 of the first embodiment. The processing circuitry 11b has a configuration in which a warning function 118 is added to the processing circuitry 11. Moreover, in the fourth embodiment, the ultrasonic diagnostic apparatus 100 is connected to a puncture position information acquisition apparatus 60.

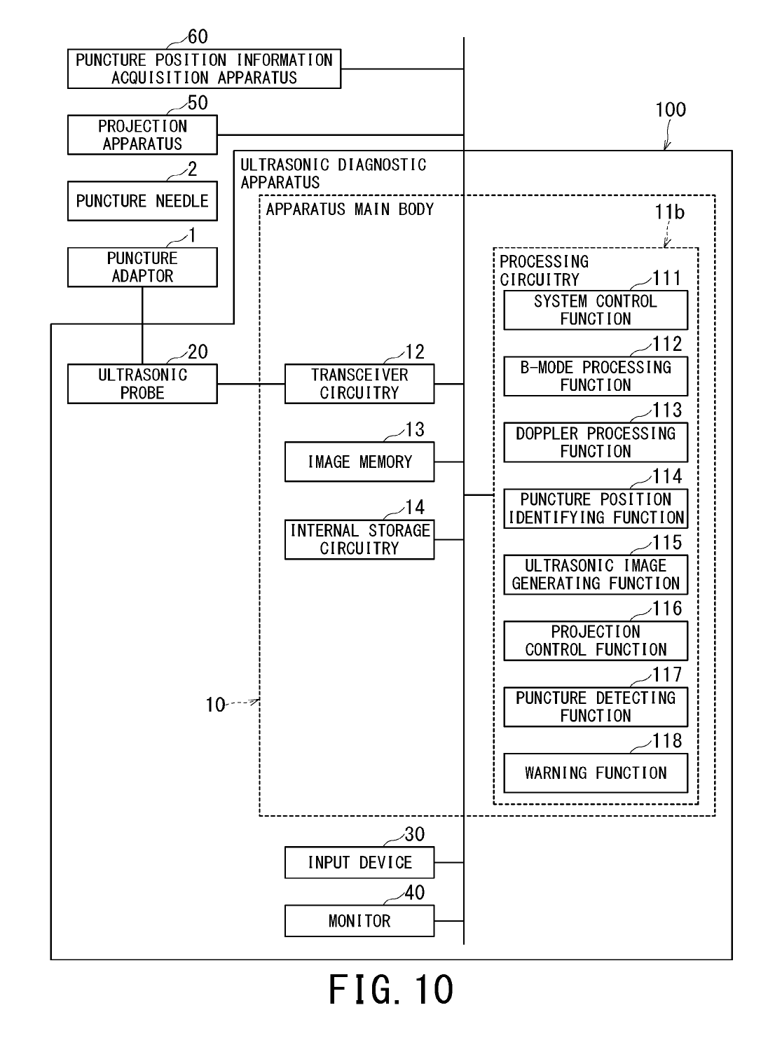

FIG. 10 is a block diagram showing an outline configuration of the ultrasonic diagnostic apparatus 100 according to the fourth embodiment. To be specific, FIG. 10 is a block diagram which extracts the processing circuitry 11b of the ultrasonic diagnostic apparatus 100 of the fourth embodiment.

The warning function 118 (warning section) has a function of notifying of a warning the operator when the puncture needle 2 is inserted into a puncture hole other than the one identified by the puncture position identifying function 114. First, the puncture detecting function 117 detects whether or not the puncture needle 2 is inserted into the desired puncture hole 31, via the puncture position information acquisition apparatus 60 to be described later. The warning function 118 makes warning upon detection that the puncture needle 2 is inserted into a different puncture hole 31.

The puncture position information acquisition apparatus 60 (puncture position information acquisition section) is made up of, for example, an optical sensor which photographs an optical image of the puncture needle 2. The puncture position information acquisition apparatus 60 detects, on the optical image, whether or not the puncture needle 2 is inserted into the desired puncture hole 31 which is being projected. Moreover, when the puncture position information acquisition apparatus 60 is an optical sensor, an infrared camera and a video camera apply thereto. The puncture position information acquisition apparatus 60 may be made integral with the projection apparatus 50, or separately therefrom, and may be attached to a position different from that of the projection apparatus 50. Moreover, the puncture position information acquisition apparatus 60 may utilize a position sensor in place of the optical sensor. In this situation, the puncture position information acquisition apparatus 60 which is made up of the position sensor is attached to the puncture needle 2, and outputs the position information of the puncture needle 2 to the puncture detecting function 117.

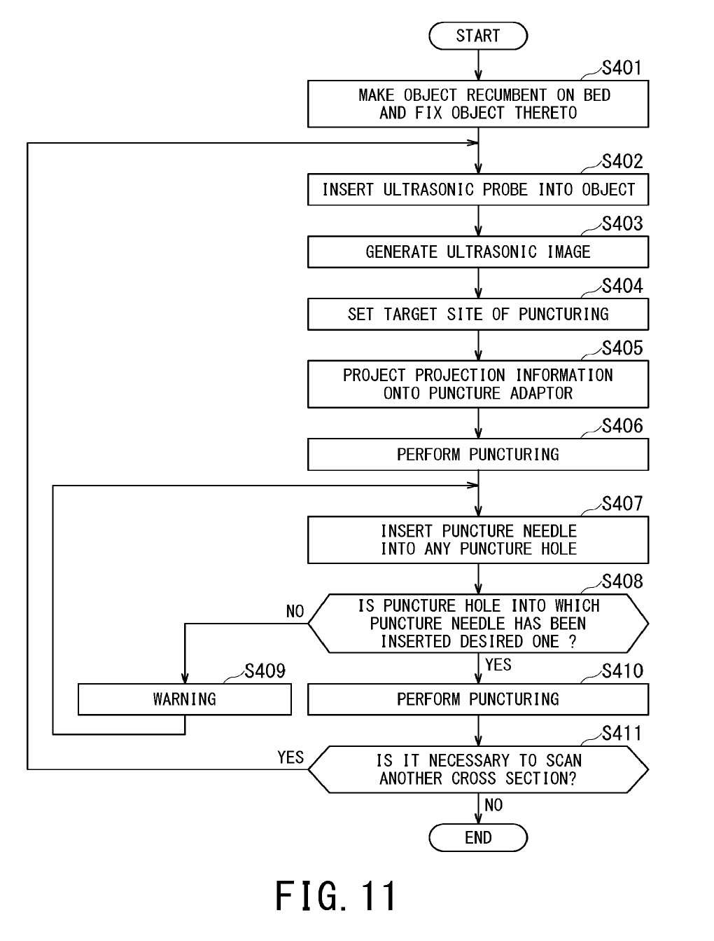

FIG. 11 is a flowchart showing an example of warning notification by the ultrasonic diagnostic apparatus 100 according to the fourth embodiment.

Regarding the present flowchart, although description will be made on the configuration in which the fourth embodiment is added to the first embodiment, the fourth embodiment may be added to any of the embodiments.

Since the processing according to from S401 to S406 is of the same configuration as the processing according to from S101 to S107, description thereof will be omitted.

First, at S407, the operator inserts the puncture needle 2 into any puncture hole 31 on the puncture adaptor 1.

At S408, it is detected whether or not the puncture needle 2, which has been inserted by the operator at S407, is inserted into a desired puncture hole 31. To be more specific, the puncture detecting function 117 optically detects whether or not the puncture needle 2 has been inserted into the desired puncture hole 31 which is being projected via the puncture position information acquisition apparatus 60. In this situation, for example, the distance in projection information or the like between the puncture needle 2 and the puncture hole 31 is detected on an optical image, and when they are spaced apart not less than a fixed distance, it is detected that the puncture position of the puncture needle 2 is displaced. The operator confirms that the inserted puncture needle 2 has been inserted into the desired puncture hole 31, and performs puncturing at S410. On the other hand, when the puncture needle 2 has been inserted into a puncture hole 31 different from the desired puncture hole 31, the process proceeds to S409.

At S409, when the puncture needle 2 is inserted into a puncture hole 31 different from the desired puncture hole 31, a warning is sent. The warning function 118 receives information regarding displacement between the puncture needle 2 and the desired puncture hole 31 from the puncture detecting function 117, and performs warning. As a warning method, for example, warning information may be projected on the puncture adaptor 1 via the projection apparatus 50, or a warning screen may be displayed on the monitor 40. Moreover, warning may be performed by voice. The operator refers to warning information, and returns to S407 again to insert the puncture needle 2 into the puncture hole 31.

At S410, when the puncture position identifying function 114 detects that the puncture needle 2 has been inserted into the desired puncture hole 31, puncturing is performed by the operator. Since the processing according to S411 is the same as that of S107, description thereof will be omitted.

By performing a series of above described processing, when the puncture needle 2 is inserted into a puncture hole 31 different from the desired puncture hole 31, the ultrasonic diagnostic apparatus 100 is able to perform warning, thus preventing failure of puncturing. Moreover, in the fourth embodiment, although description has been made on a case in which when the puncture needle 2 is inserted into a puncture hole 31 different from the desired puncture hole 31, warning display is performed, the timing of warning display will not be limited to the above described one. For example, it has been described in the first embodiment that the puncture detecting function 117 detects that puncturing has been completed when a radiation source is disposed at a target site of puncturing on an ultrasonic image. Similarly, it may be arranged that when a radiation source and the puncture needle 2 are not detected after insertion of the puncture needle 2 into the puncture adaptor 1 at the target site of puncturing by the puncture detecting function 117, the warning function 118 performs warning display.

### 5. Fifth Embodiment

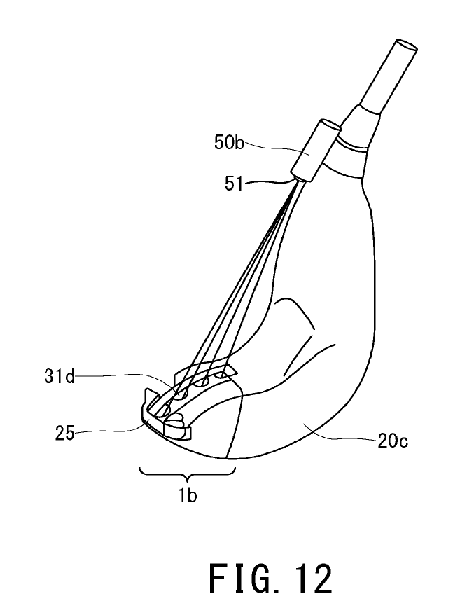

In the first to fourth embodiments, description has been made taking example of the puncture adaptor 1 to be used during brachytherapy of a prostate cancer. Next, in the fifth embodiment, description will be made by using FIG. 12 on an ultrasonic diagnostic apparatus 100 in which a puncture adaptor 1b and an ultrasonic probe 20c are integrated with each other.

FIG. 12 is a diagram showing an example of a puncture adaptor and an ultrasonic probe included in the ultrasonic diagnostic apparatus 100 according to a fifth embodiment.

The puncture adaptor 1b in the fifth embodiment is configured to be detachable via an ultrasonic probe 20c and a fixture 25. A projection apparatus 50b (projection section) in the fifth embodiment is made up of, for example, a laser pointer 51 attached to the ultrasonic probe 20c and a small size projector. The configuration is such that the projection apparatus 50b is attached to the ultrasonic probe 20c, and the laser pointer 51 is attached to the projection apparatus 50b. The laser pointer 51 may be provided in the number corresponding to that of the puncture holes 31d, or only the desired puncture hole 31d may be irradiated with a laser by varying the irradiation angle of one laser pointer 51. As a result of this laser pointer 51 irradiating a desired puncture hole 31d with laser, the operator is able to identify the desired puncture hole 31d.

The projection apparatus 50b will not be limited to the configuration in which it is attached to the ultrasonic probe 20c, and the configuration may be such that the projection apparatus 50b is separately provided. When the projection apparatus 50b is provided, a marker, etc. for alignment with the projection apparatus 50b is put on the puncture adaptor 1b, and the position of the puncture hole 31d is identified by the projection apparatus 50b recognizing the marker position.

When the puncture adaptor 1 has a structure in which a plane including the puncture holes 31 has irregularities, a projector capable of 3D mapping may be used as the projection apparatus 50b. Examples of the structure including irregularities include, for example, a structure in which the plane of the puncture adaptor 1 is curved. 3D mapping is a technique to portray a projection image created by computer on an object or in a space by using a projector, thus following the movement of a subject. When performing 3D mapping, it is necessary to recognize the shape of the puncture adaptor by reading a marker, etc. on the puncture adaptor 1 with a photographing apparatus such as a camera, and change the projection direction of the projection information. The photographing apparatus such as a camera is one example of a puncture adaptor recognition section for recognizing the shape of the puncture adaptor.

The resolution of display image of the projector may be enhanced by disposing a screen so as to cover the puncture adaptor 1, and projecting projection information onto the screen.

In the embodiments described so far, although description has been made assuming that the ultrasonic diagnostic apparatus 100 includes an ultrasonic probe 20, the ultrasonic probe 20 may have a configuration separate from the ultrasonic diagnostic apparatus 100. Moreover, although the projection apparatus 50 and the puncture position information acquisition apparatus 60 in the fourth embodiment have been described as bodies separate from the ultrasonic diagnostic apparatus 100, they may be configured to be integrated with the ultrasonic diagnostic apparatus 100.

### 6. Effects

According to embodiments described so far, it becomes possible to project and display the position of a desired puncture hole 31 onto the puncture adaptor 1 by using the projection apparatus 50. This makes it possible that the operator refers to projection information indicating puncture position displayed on the puncture adaptor 1 and performs puncturing into the desired puncture hole 31, thus reducing the possibility that the operator mistakes the position of the desired puncture hole 31.

The configuration in which a puncture plan is, as the projection information, projected onto the puncture adaptor 1 allows to make the puncture adaptor 1 disposable without needing a power supply, wiring and the like associated with the puncture adaptor 1. This makes it unnecessary to sterilize and clean the puncture adaptor at every puncture treatment, thus allowing to mitigate the load on the operator.

Use of a 3D mapping apparatus for the projection apparatus 50 allows to project projection information regarding puncturing without depending on the outer shape of the puncture adaptor 1. Further, the warning function 118 allows to reduce the possibility that the operator inserts the puncture needle 2 into a wrong puncture hole 31.

The ultrasonic diagnostic apparatus 100 is configured such that the puncture adaptor 1 itself does not issue information, but the projection apparatus disposed at a position spaced apart from the puncture adaptor 1 issues information. The ultrasonic diagnostic apparatus 100 allows to present projection information every time during surgery even if only the puncture adaptor 1 which is likely to be contaminated by puncturing is made disposal. Therefore, the ultrasonic diagnostic apparatus 100 allows to keep the puncture adaptor 1 always hygienic.

Note that regarding any component which is herein described as a “section”, its action may be implemented by hardware, or by software, or by a combination of hardware and software.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

What is claimed is:

1. An ultrasonic diagnostic apparatus, comprising:
an ultrasonic probe attached to a puncture adaptor including puncture holes; and
a processing circuitry configured to
(A) set a target site of puncturing within an object, the target site of puncturing being specified in an ultrasonic image based on data detected by the ultrasonic probe,
(B) identify a puncture hole out of the puncture holes, the puncture hole being at a position corresponding to a position of the target site of puncturing,
(C) generate projection information regarding the identified puncture hole, and
(D) control a projection apparatus that optically projects information to control the projection information to be projected onto the puncture adaptor.

2. The ultrasonic diagnostic apparatus according to claim 1, wherein
the processing circuitry is configured to:
identify position information of a puncture needle, and
perform warning based on the identified position information of the puncture needle.

3. The ultrasonic diagnostic apparatus according to claim 1, wherein
the processing circuitry is configured to identify, out of the puncture holes, a puncture hole at a position whose distance from the position of the target site of puncturing specified in the ultrasonic image is shortest.

4. The ultrasonic diagnostic apparatus according to claim 1, wherein
the processing circuitry is configured to specify the projection information to be two or more line segments which include the puncture hole corresponding to the target site of puncturing in the puncture adaptor, and which intersect at the puncture hole.

5. The ultrasonic diagnostic apparatus according to claim 1, wherein
the processing circuitry is configured to specify the projection information to be the ultrasonic image including the target site of puncturing.

6. The ultrasonic diagnostic apparatus according to claim 5, wherein
the processing circuitry is configured to specify the projection information to be a real time image of the ultrasonic image including the target site of puncturing.

7. The ultrasonic diagnostic apparatus according to claim 1, wherein
the processing circuitry is configured to specify the projection information, when the target site of puncturing is target sites of puncturing, to be information for simultaneously projecting puncture holes at positions respectively corresponding to positions of the target sites of puncturing.

8. The ultrasonic diagnostic apparatus according to claim 1, wherein
the processing circuitry is configured to specify the projection information, when the target site of puncturing is target sites of puncturing, to be information for successively projecting puncture holes at positions respectively corresponding to positions of the target sites of puncturing.

9. The ultrasonic diagnostic apparatus according to claim 1, wherein
the processing circuitry is configured to specify the projection information to be projection information regarding the puncture hole, and also to be information for emphasizing and projecting the puncture hole at which puncturing is finished onto the puncture adaptor.

10. The ultrasonic diagnostic apparatus according to claim 1, wherein
the processing circuitry is configured to specify the projection information to be projection information regarding the puncture hole, and also to be information for projecting a number of times of puncturing onto the puncture adaptor.

11. The ultrasonic diagnostic apparatus according to claim 1, further comprising
the projection apparatus, wherein
the projection apparatus is a projector configured to recognize a shape of the puncture adaptor, and to change, depending on the shape of the puncture adaptor, a shape of the projection information to project the shape.

12. The ultrasonic diagnostic apparatus according to claim 1, further comprising
the projection apparatus, wherein
the projection apparatus is a laser pointer for pointing at the puncture hole.

13. The ultrasonic diagnostic apparatus according to claim 2, further comprising
a puncture adaptor including a marker for performing alignment with the projection information.

14. The ultrasonic diagnostic apparatus according to claim 5, wherein
the ultrasonic probe is able to execute 3-dimensional scanning for acquiring volume data of the object, and
the processing circuitry is configured to generate a reconstructed image in an arbitrary cross section of the volume data such that the projection information is the reconstructed image.

15. The ultrasonic diagnostic apparatus according to claim 14, further comprising
a puncture position information acquisition apparatus configured to identify position information of a puncture needle, wherein
the processing circuitry is configured to specify the arbitrary cross section to be a cross section corresponding to the position information of the puncture needle.

16. The ultrasonic diagnostic apparatus according to claim 15, wherein
the puncture position information acquisition apparatus is configured to be integral with the projection apparatus.

17. The ultrasonic diagnostic apparatus according to claim 15, wherein
the puncture position information acquisition apparatus is a photographing apparatus of an optical image and configured to acquire the position information of the puncture needle based on the optical image.

18. The ultrasonic diagnostic apparatus according to claim 15, wherein
the puncture position information acquisition apparatus is a position sensor configured to detect the position information of the puncture needle.

19. The ultrasonic diagnostic apparatus according to claim 1, further comprising
the puncture adaptor, wherein
the puncture adaptor and the ultrasonic probe are configured to be integral with each other, and
the projection apparatus is attached to the ultrasonic probe and configured to project the identified puncture hole of the puncture adaptor.
